(19) United States
(12) Patent Application Publication
(10) Pub. No.: US 2012/0029384 A1
(43) Pub. Date: Feb. 2, 2012

(54) Accutainer

(76) Inventor: Jay Clifford Crosman, Mamaroneck, NY (US)

(21) Appl. No.: 12/804,741

(22) Filed: Jul. 28, 2010

(51) Int. Cl. A61B 5/153

(52) U.S. Cl. 600/577

(57) ABSTRACT

This invention is a device which can be used to obtain a sample of blood from a person or animal. It incorporates parts of two existing devices (syringes and Vacutainers) into one device in a new configuration, and it reduces or eliminates some of the disadvantages of each of these two existing devices. Unlike the typical use of a syringe, a sample of blood is transferred directly from the blood vessel of a person or animal to the test tube or similar container that is sent to a laboratory for testing. Unlike a Vacutainer, the differential pressure between the two ends of a hypodermic needle can be controlled while the device is being used.

### DESCRIPTION OF THE ATTACHED FIGURES

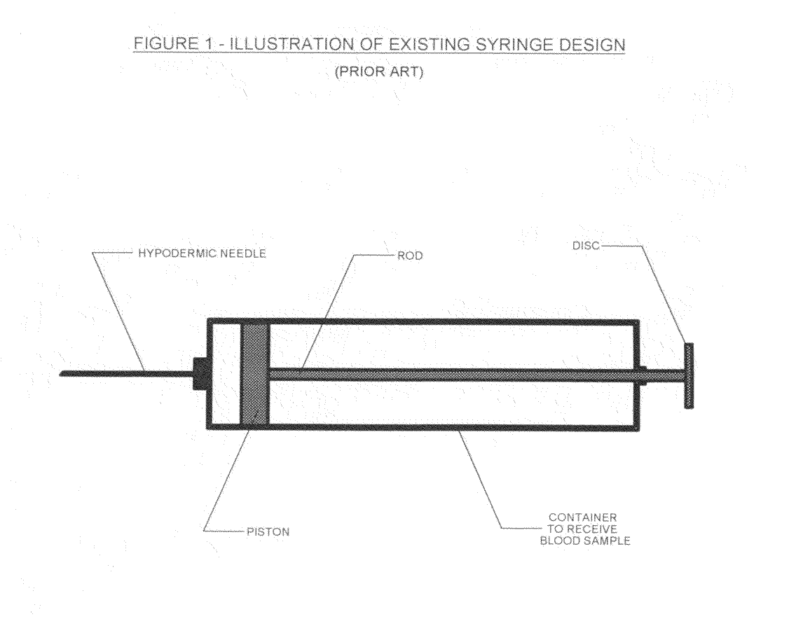

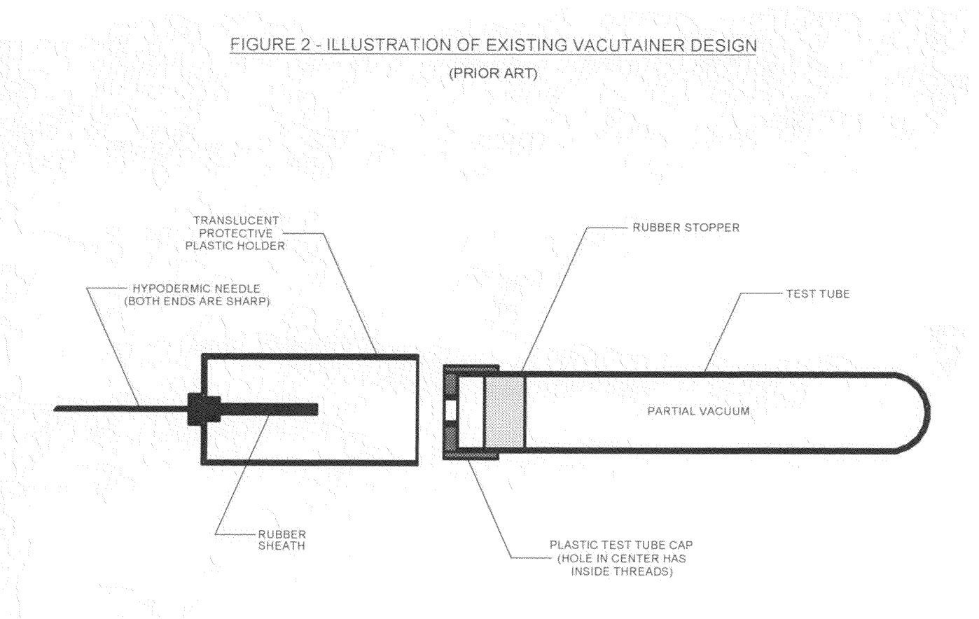

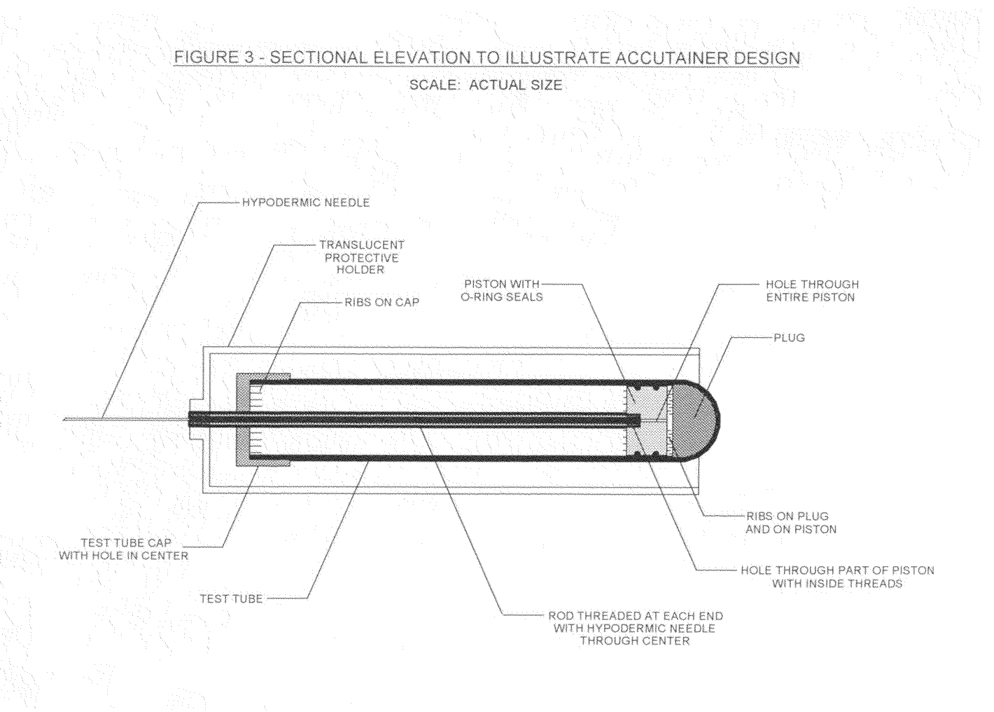

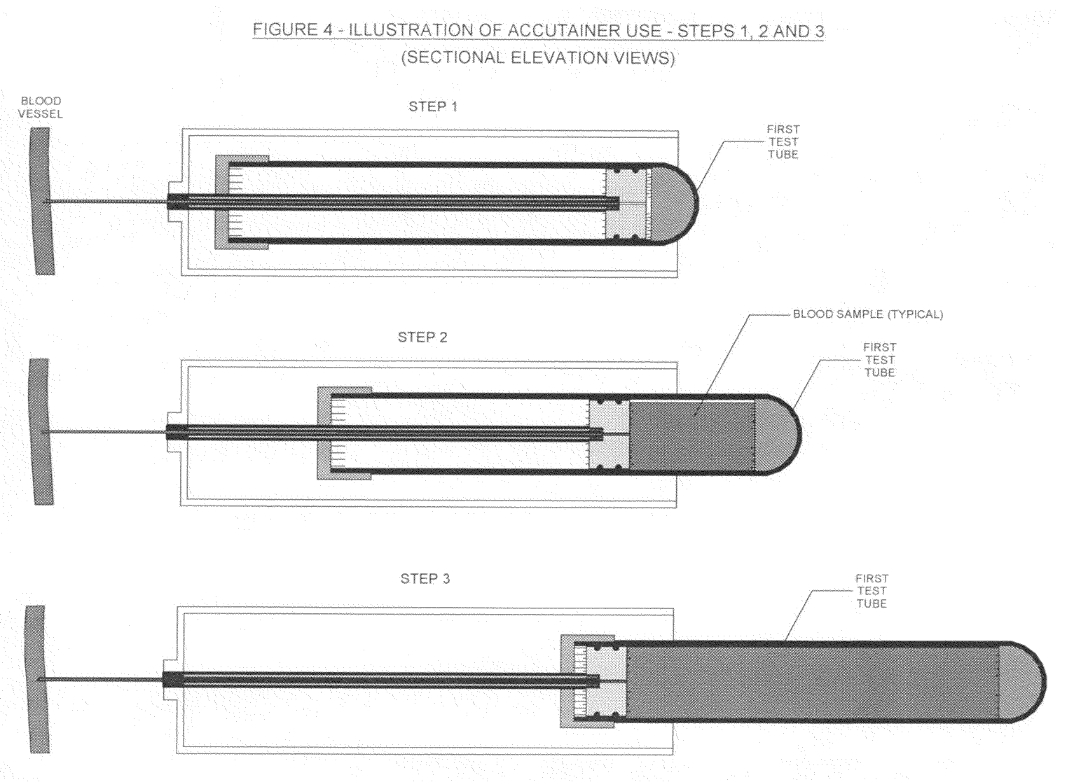

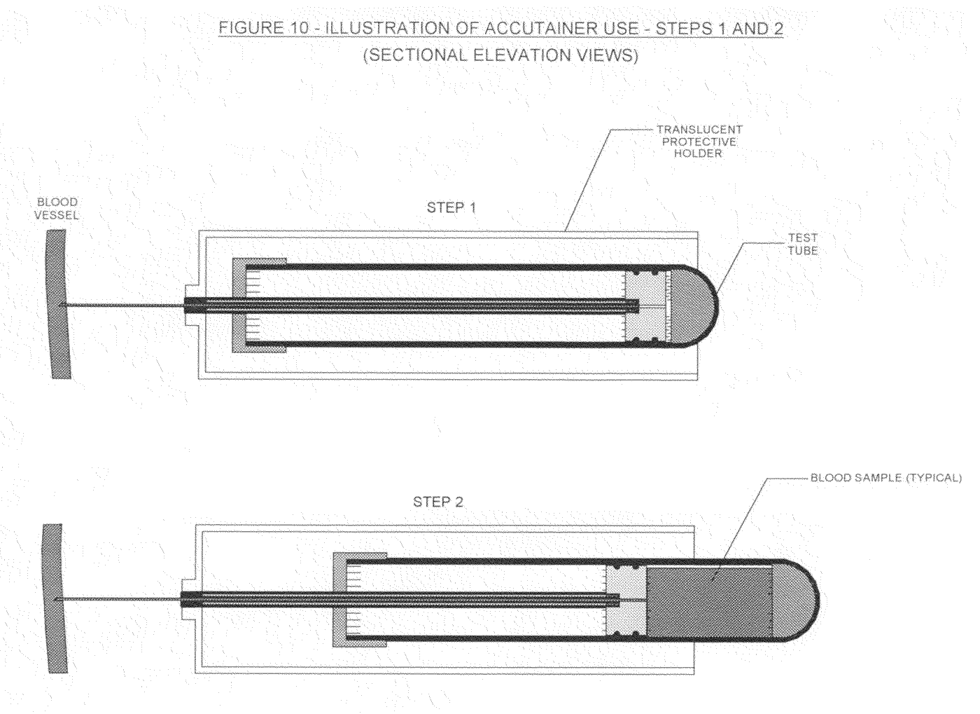

The design of a syringe is illustrated in FIG. 1 and the design of a Vacutainer is illustrated in FIG. 2. The preferred embodiment of the invention is illustrated in FIG. 3. How the preferred embodiment can be used is illustrated in FIGS. 4, 5, 6, 7, 8 and 9. An additional method of how the preferred embodiment can be used is illustrated in FIGS. 10, 11, 12, 13, 14, and 15.

### BACKGROUND OF THE INVENTION

A syringe, as illustrated in FIG. 1, can be used to obtain a sample of blood from a person or animal.

The hypodermic needle is pushed through the skin of the person or animal and into a blood vessel. A person using it holds the container with one hand and pulls the disc or handle to the right with the other hand. This causes the piston to also move to the right. When the piston is moved to the right, the volume inside the cylinder is increased and the pressure of gas enclosed in the portion of the container to the left of the piston is decreased. The differential pressure between the two ends of the hypodermic needle is increased causing blood to flow from the blood vessel of a person or animal through the hypodermic needle and into the portion of the container to the left of the piston. The entire device is then pulled to the right and the hypodermic needle is removed from the person or animal.

The blood sample now in the container must generally be transferred to a test tube so that it can then be sent to a laboratory for testing. This can be accomplished by orienting the syringe in a vertical position, putting the end of the hypodermic needle into a test tube that is also in a vertical position, and pushing the disc downward. Pushing the disc downward forces the sample of blood out though the end of the hypodermic needle and into the test tube.

An advantage of using a syringe is that person using it can control the differential pressure across the two ends of the hypodermic needle by changing the speed at which the disc or handle is moved. A person using it has some control over the flow of blood from a blood vessel into the syringe. An additional advantage is that after the hypodermic needle has been removed from the person or animal, a person using it can pull the disc or handle farther to the right. This may partially or completely evacuate the hollow core of the needle. It can prevent some of the sample of blood from escaping from the left end of the hypodermic needle, and thereby reduce the possibility that a person using the syringe will be exposed to blood that could potentially contain pathogens.

A disadvantage of using a syringe is that a sample of blood is generally transferred twice. It is first transferred from a blood vessel to the container of the syringe, and then from the syringe to a test tube. With two transfers, there is a greater potential for the composition of a blood sample to change than with one transfer. With two transfers, there is also a greater potential for some of the blood sample to not be entirely contained within the syringe or test tube. There is a greater potential for a person using the syringe and transferring the sample to a test tube to be exposed to blood that may contain pathogens.

An additional disadvantage of using a syringe is that the hypodermic needle would generally need to be pushed into the blood vessel each time a sample is obtained. If multiple samples of blood are needed, the hypodermic needle would generally need to be put into the blood vessel and taken out several times. This is likely to increase the pain or discomfort experienced by the person or animal from which the sample is obtained.

A Vacutainer, as illustrated in FIG. 2, can also be used to obtain a sample of blood from a person or animal.

The hypodermic needle is pushed through the skin of the person or animal and into a blood vessel. A person using it holds the container with one hand and pushes the test tube to the left. The right side of the needle is encased in a rubber sheath and is sharpened. Pushing the test tube to the left causes the right side of the hypodermic needle to pierce the rubber stopper and come into contact with the partial vacuum inside the test tube on the opposite side of the rubber stopper.

After the test tube is manufactured, a partial vacuum (absolute pressure that is below atmospheric pressure) exists inside the test tube to the right of the rubber stopper. This partial vacuum is maintained until the test tube is used because the rubber stopper prevents air from entering the test tube.

Because the pressure inside a blood vessel is greater than the pressure of the partial vacuum inside the test tube, blood flows from the blood vessel through the hypodermic needle into the test tube. As blood flows into the test tube, the volume of the space occupied by gas will decrease and the pressure will therefore increase. When the pressure of gas inside the test tube is equal to the pressure at the opposite end of the hypodermic needle (the end in the blood vessel), blood will cease to flow through the hypodermic needle.

During this procedure, a person using the device cannot control the rate of blood flow through the hypodermic needle. The rate of blood flow is initially determined by difference between the blood pressure in the blood vessel and the initial degree of vacuum that exists after the test tube is manufactured. The differential pressure decreases from its initial value to essentially zero as blood flows into the test tube.

After the sample of blood is collected in the test tube, the test tube is moved to the right, and the right side of the hypodermic needle is thereby removed from the rubber stopper. The test tube with the sample of blood in it can then be sent to a laboratory for testing.

While the left end of the hypodermic needle remains in the blood vessel of a person or animal, additional samples of blood can be obtained by using additional test tubes. After all of the blood samples have been transferred to test tubes, the translucent protective holder is moved to the right and the left end of the hypodermic needle is removed from the person or animal.

An advantage of a Vacutainer in comparison to a syringe is that a sample of blood is only transferred once. It is transferred directly into a test tube that is then sent to a laboratory. With only one transfer, the potential for a person using the device to be exposed to blood that could contain pathogens is reduced.

An additional advantage of a Vacutainer is that more than one sample of blood can be obtained even though the hypodermic needle is only pushed through the skin and into the blood vessel once. A series of test tubes can be used and each test tube can be different. One test tube could contain a chemical to minimize blood clotting and another could contain no chemical.

A limitation of a Vacutainer is that a person using it cannot control the differential pressure at the two ends of the hypodermic needle while the device is being used. The initial differential pressure between the two ends of the hypodermic needle is the difference between the pressure inside the blood vessel at the left end of it and the initial degree of vacuum inside the test tube.

Since the differential pressure across the two ends of the hypodermic needle cannot be controlled while the device is being used, the flow rate of blood through the hypodermic needle also cannot be controlled while the device is being used. If test tubes with the same degree of vacuum are used to obtain samples of blood from many different people, the flow rate of blood through the hypodermic needle may be acceptable for some people, but could cause a vein to collapse in others.

The blood pressure in one person may be different than another. If test tubes manufactured with one specific degree of vacuum are used on two different people, the initial differential pressure across the two ends of the hypodermic needle may be different. A person using the device may not be able to assess what initial degree of vacuum is most appropriate for a particular person from whom the blood sample is to be taken.

An additional limitation of a Vacutainer is that a person using the device cannot control the volume of blood sample obtained in a test tube. The volume of gas inside the test tube when the blood ceases to flow is determined by its pressure which will be essentially equal to the pressure of blood inside the blood vessel from which the sample is taken. Since the blood pressure in one person may be different than another, this volume of gas may be different. Since the volume of gas may be different, the volume of blood collected in the test tube may also be different.

Since the volume of sample collected may be different when the device is used on several people, the quantity of a chemical initially inside the test tube may be correct when the blood sample is collected from some people, but not others.

When the hypodermic needle is removed from a person or animal, the hollow core of it is filled with blood. This blood will be exposed to air at a lower pressure than the pressure of the blood vessel it had been in contact with. If the sample has been taken from a vein, carbon dioxide is likely to come out of solution, and the blood is likely to absorb oxygen. This change of pressure and transfer of gases could cause a change in the volume of the blood in the hollow core of the hypodermic needle, and cause some of the blood to escape from one end of it. If this occurs, a person using the device could be exposed to small quantity of blood that may contain pathogens.

### SUMMARY OF THE INVENTION

An object of the invention is to provide one device that will allow a user of it to transfer a sample of blood from the blood vessel of a person or animal directly to a test tube or similar container that will be sent to a laboratory for testing of the sample, obtain samples in two or more test tubes or similar containers while only pushing the hypodermic needle into the blood vessel of a person or animal once, control the differential pressure between the two ends of a hypodermic needle, and obtain a more consistent or repeatable volume of sample when the device is used to obtain samples from several different people or animals.

The accomplishment of this object results in a device that is safer for a person using it, more adaptable to the needs of different people or animals from whom a sample of blood is being taken; and it permits a user of the device to obtain a more accurate volume of blood than devices which have previously been used.

### DETAILED DESCRIPTION OF THE PREFERRED EMBODIMENT

FIG. 3 is a sectional elevation view of the preferred embodiment of the invention.

As illustrated in FIG. 3, the preferred embodiment of the invention includes a hypodermic needle partially within and attached to a plastic rod that is threaded at each end. The left end of the hypodermic needle is sharpened and extends beyond the left end of the plastic rod. The right end of the hypodermic needle extends to the right end of the plastic rod. The hypodermic needle has a hollow core and is open at both ends. The left end of the plastic rod is threaded into a translucent protective holder. The left end is threaded tightly enough so that it cannot easily be removed by rotation. An adhesive could be used to accomplish this. The right end of the plastic rod is threaded into a piston located inside a test tube.

The outside diameter of the piston is less than the inside diameter of the test tube. It has one or more O-rings to prevent or minimize the flow of gas or liquid between its left and right sides. The O-rings are designed to allow the test tube to move to the right or left while the piston remains in a fixed position. The piston includes a hole partially through its length with inside threads to allow the plastic rod to be threaded into it. It includes a hole completely through to its right side that is approximately the same diameter as the hollow core of the hypodermic needle. When the plastic rod is threaded into the piston, the left end of the hypodermic needle is thereby open to the right side of the piston. The left and right sides of the piston have ribs across the surface.

The test tube has a cap with a hole in its center. The cap is attached and/or fitted to the test tube tightly enough so that the cap rotates when the test tube is rotated axially. The hole is large enough to allow the plastic rod to be pushed through it. The hole is large enough to allow the test tube and cap to move to the right or left. The hole is small enough to keep the test tube essentially centered on the rod when it is moved to the right or left.

This invention could be designed to use containers similar to test tubes with an open top, closed sides, and a closed base. When a test tube is oriented vertically, a horizontal cross section has a circular shape. Similar containers could have elliptical, rectangular, or other horizontal cross sectional shapes when oriented in the same way. The piston and O-rings could be designed to operate inside similar containers with a horizontal cross sectional shapes other than circular.

The right side of the cap, the side facing the inside of the test tube, has ribs running across its surface. The ribs located on the left side of the piston and the ribs located on the right side of the cap are designed to make the piston rotate when it is close enough to the cap and when the test tube and cap are rotated around a horizontal axis. Other methods of making the two rotate together are possible.

Within the test tube at the right end is a plug. The plug is fitted tightly enough to the inside of the test tube so that it rotates when the test tube is rotated. The left side of the plug has ribs across its surface. The ribs are designed to make the piston rotate when it is close enough to the plug and when the plug and test tube are rotated around a horizontal axis.

By means of the above, the right end of the threaded rod can be threaded into the piston when it is near the plug. The threaded rod can also be unthreaded from the piston when it is close to the cap of the test tube.

### DETAILED DESCRIPTION OF HOW THE PREFERRED EMBODIMENT CAN BE USED

FIGS. 4, 5, 6, 7, 8, and 9 illustrate how the preferred embodiment can be used.

The left end of the hypodermic needle is pushed through the skin of a person or animal and into a blood vessel as shown in FIG. 4, Step 1.

A person using the device then holds the translucent protective holder in place while pulling the test tube to the right as illustrated in FIG. 4, step 2,. Since a fixed amount of gas will now be located within a larger volume, the pressure inside the portion of the test tube to the right of the piston will decrease, the differential pressure between the two ends of the hypodermic needle will increase, and blood will flow from the blood vessel through the hypodermic needle into the test tube. The differential pressure can be controlled by moving the test tube more quickly or more slowly. This will allow a person using the device to produce a higher or lower flow rate of blood into the test tube.

Step 3 of FIG. 4 illustrates the point at which the full volume of sample has been obtained. Since only a small amount of gas was originally in the test tube, the internal volume will be mostly filled with the sample of blood. Essentially the same volume of blood sample will be obtained regardless of the blood pressure of the person from whom the sample is being taken.

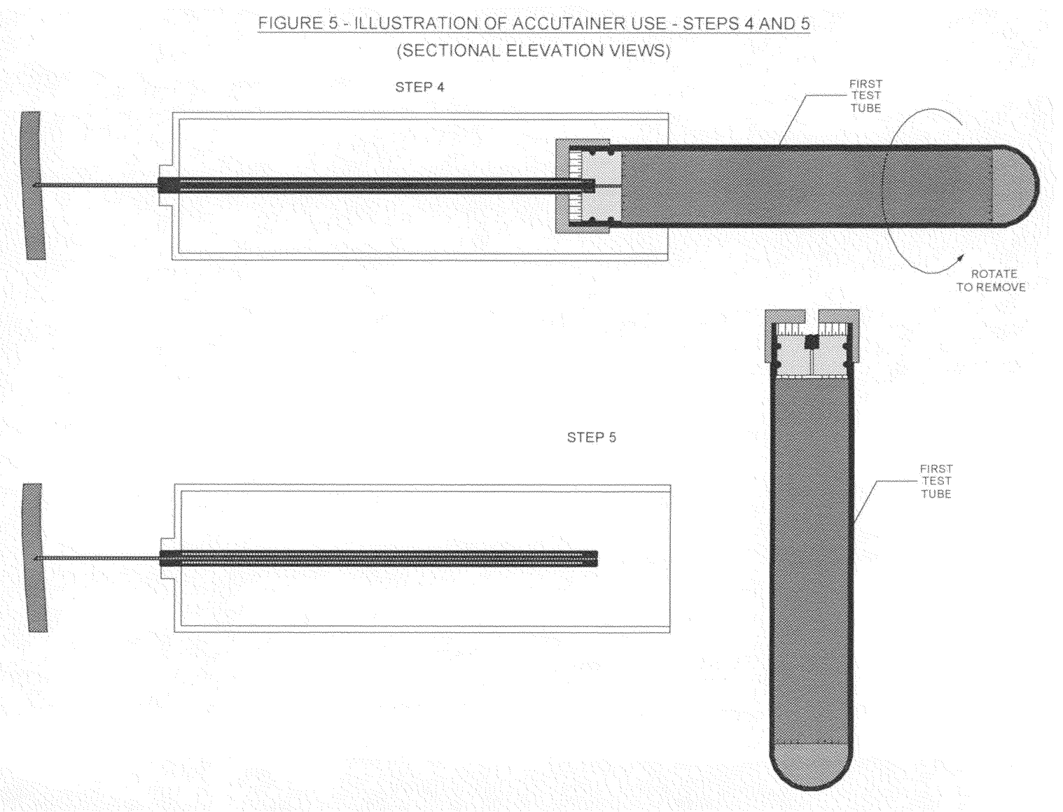

FIG. 5, step 4 illustrates unthreading the test tube from the plastic rod by rotating the test tube around a horizontal axis in the direction shown while holding the translucent protective holder in place. The ribs on the right side of the cap and the left side of the piston make the piston rotate when the test tube is rotated.

FIG. 5, step 5 shows that the test tube is now separated from the hypodermic needle and holder, and can be sent to a laboratory. The hypodermic needle can remain in the blood vessel.

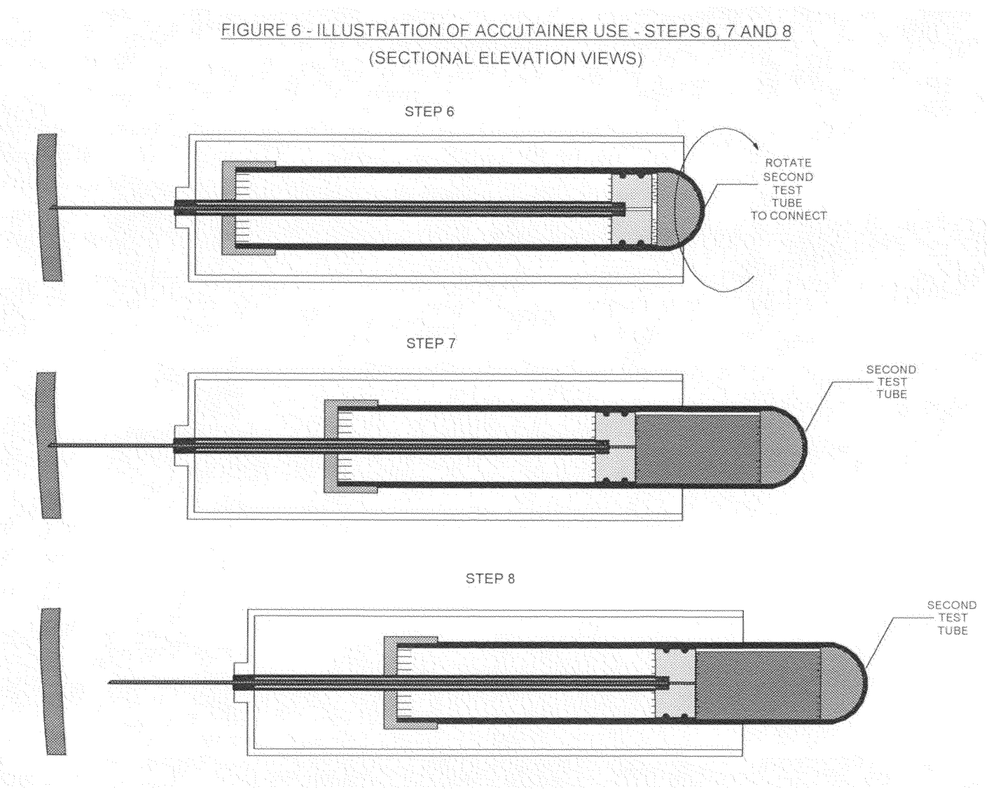

FIG. 6, step 6 illustrates how a second test tube can be threaded onto the plastic rod by holding the translucent protective holder in place and rotating the second test tube around a horizontal axis in the direction shown. The ribs on the left side of the plug make the piston rotate when the test tube is rotated.

FIG. 6, step 7 illustrates a point where some amount of sample has been obtained, but the test tube is not completely filled.

FIG. 6, step 8 illustrates that the entire device is moved to the right, and the hypodermic needle is removed from the blood vessel and skin of the person or animal from which the sample is being taken.

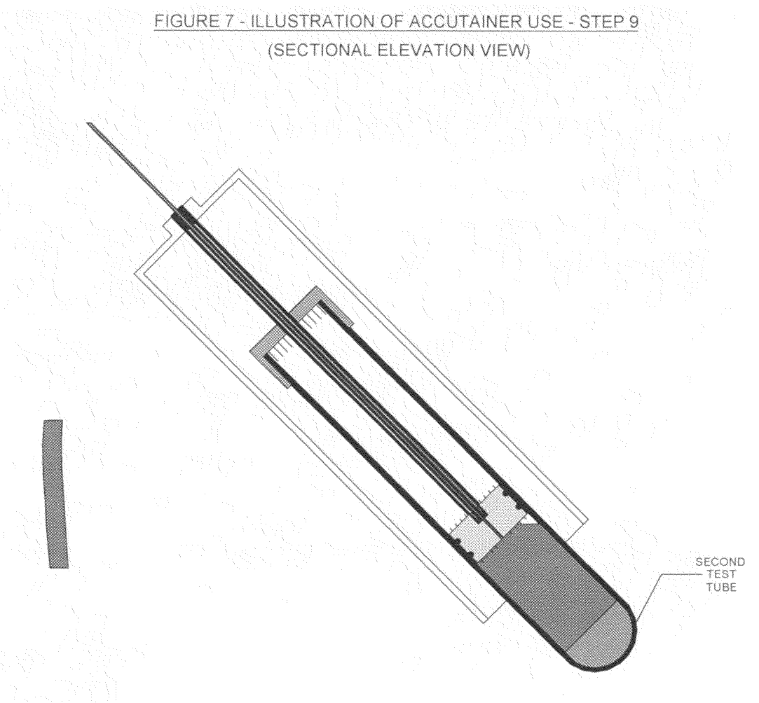

FIG. 7, step 9 illustrates that the entire device is oriented more vertically. A 45 degree angle is shown for the purpose of illustration.

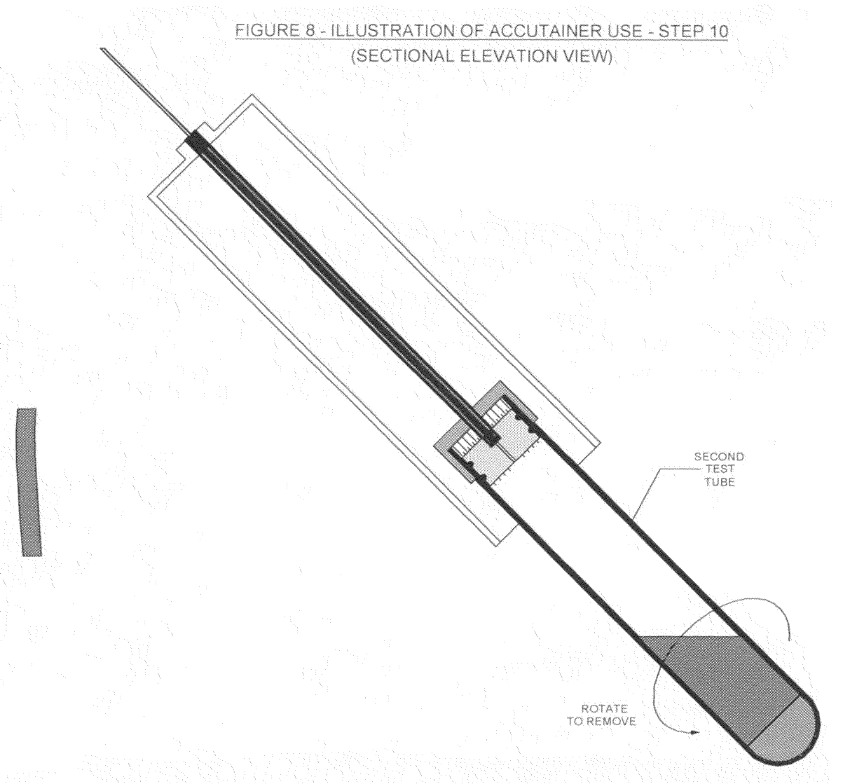

FIG. 8, step 10 illustrates that the test tube is then moved to the right and downwards. This reduces the pressure on the right end of the hypodermic needle and induces blood inside the hollow core of the hypodermic needle to flow into the test tube. It thereby reduces the potential for blood to escape from the left end of the hypodermic needle.

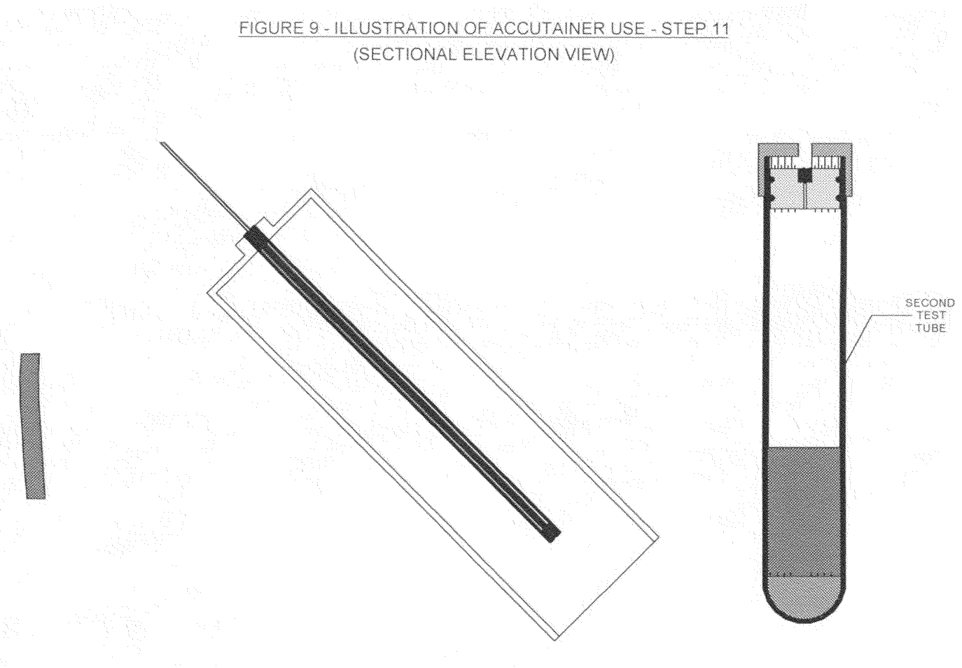

FIG. 9, step 11 illustrates that the piston in the second test tube can be unthreaded from the plastic rod in the same way as described above for the first test tube.

FIG. 10, step 11 illustrates that the second test tube is now separate and can be sent to a laboratory for testing.

The above explains how two test tubes can be filled with samples of blood while the hypodermic needle remains inside a blood vessel. Three or more test tubes could also be filled with samples of blood while the hypodermic needle remains in the blood vessel also.

### DETAILED DESCRIPTION OF AN ADDITIONAL METHOD OF USING THE PREFERRED EMBODIMENT

FIGS. 10, 11, 12, 13, 14, and 15 illustrate an additional method of using the preferred embodiment.

The left end of the hypodermic needle is pushed through the skin of a person or animal and into a blood vessel as shown in FIG. 10, Step 1.

A person using the device then holds the translucent protective holder in place while pulling the test tube to the right as illustrated in FIG. 10, step 2,. Since a fixed amount of gas will now be located within a larger volume, the pressure inside the portion of the test tube to the right of the piston will decrease, the differential pressure between the two ends of the hypodermic needle will increase, and blood will flow from the blood vessel through the hypodermic needle into the test tube. The differential pressure can be controlled by moving the test tube more quickly or more slowly. This will allow a person using the device to produce a higher or lower flow rate of blood into the test tube.

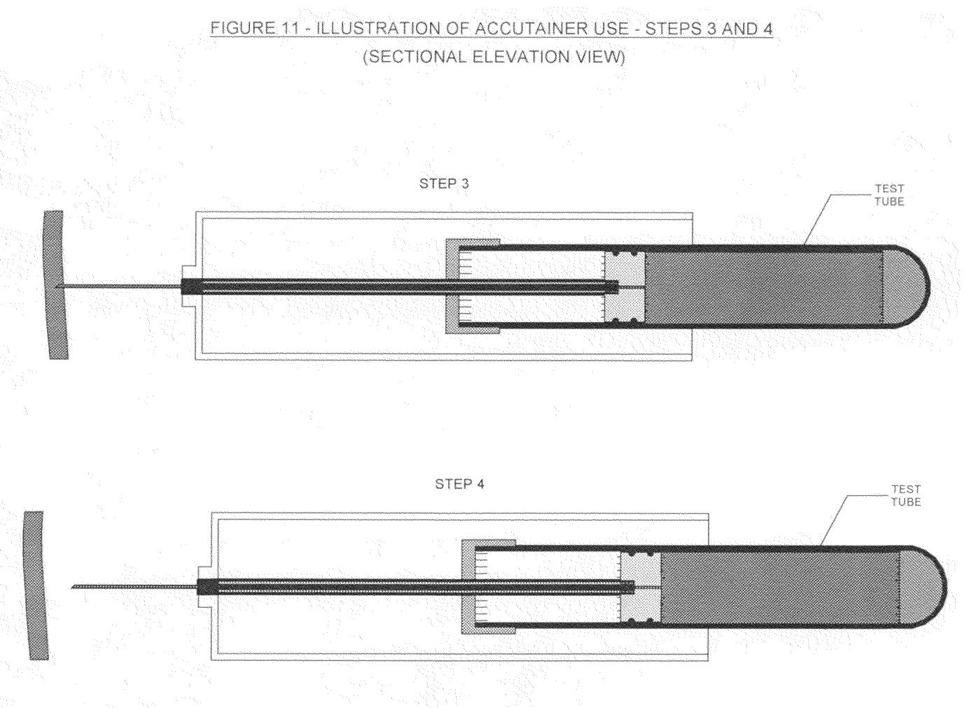

Step 3 of FIG. 11 illustrates the point at which a specific volume of sample has been obtained. The user of the device can decide to stop moving the test tube to the right before the test tube is completely filled. Since only a small amount of gas was originally in the test tube, the internal volume will be mostly filled with the sample of blood.

FIG. 11, step 4 illustrates that the entire device is moved to the right, and the hypodermic needle is removed from the blood vessel and skin of the person or animal from which the sample is being taken.

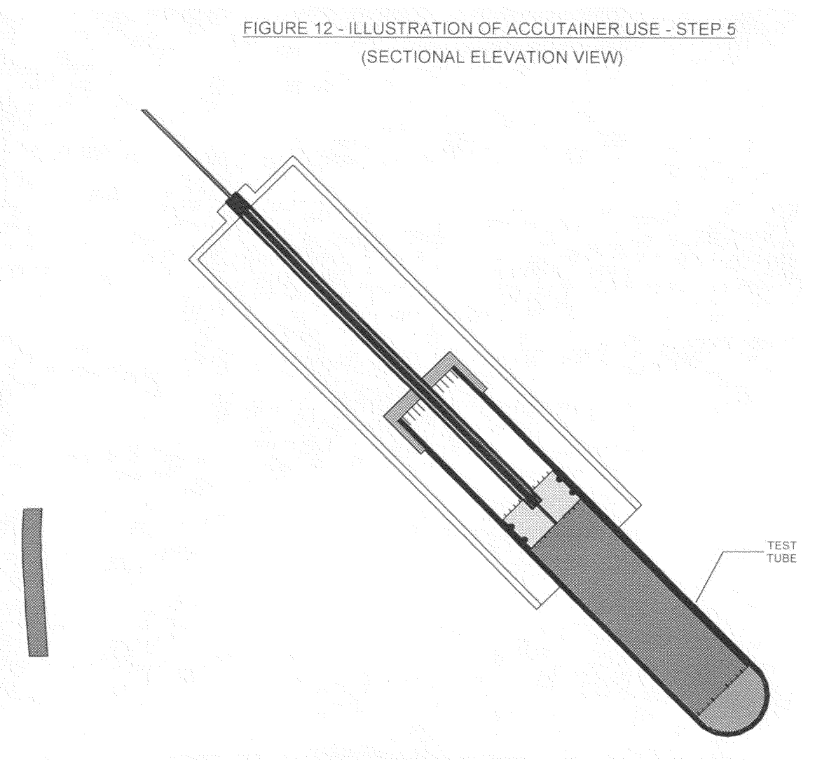

FIG. 12, step 5 illustrates that the entire device is oriented more vertically. A 45 degree angle is shown for the purpose of illustration.

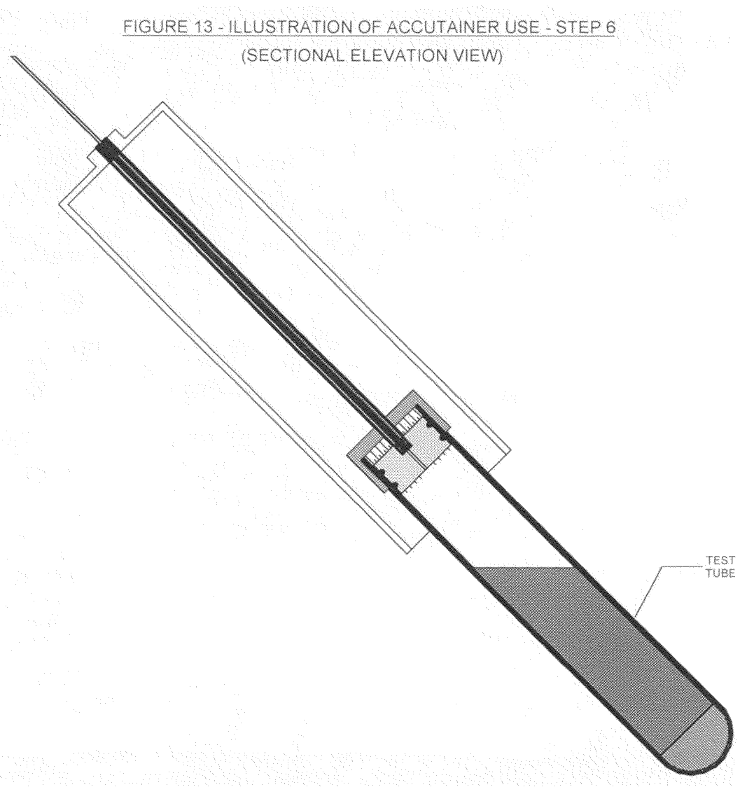

FIG. 13, step 6 illustrates that the test tube is then moved to the right and downwards. This reduces the pressure on the right end of the hypodermic needle and induces blood inside the hollow core of the hypodermic needle to flow into the test tube. It thereby reduces the potential for blood to escape from the left end of the hypodermic needle.

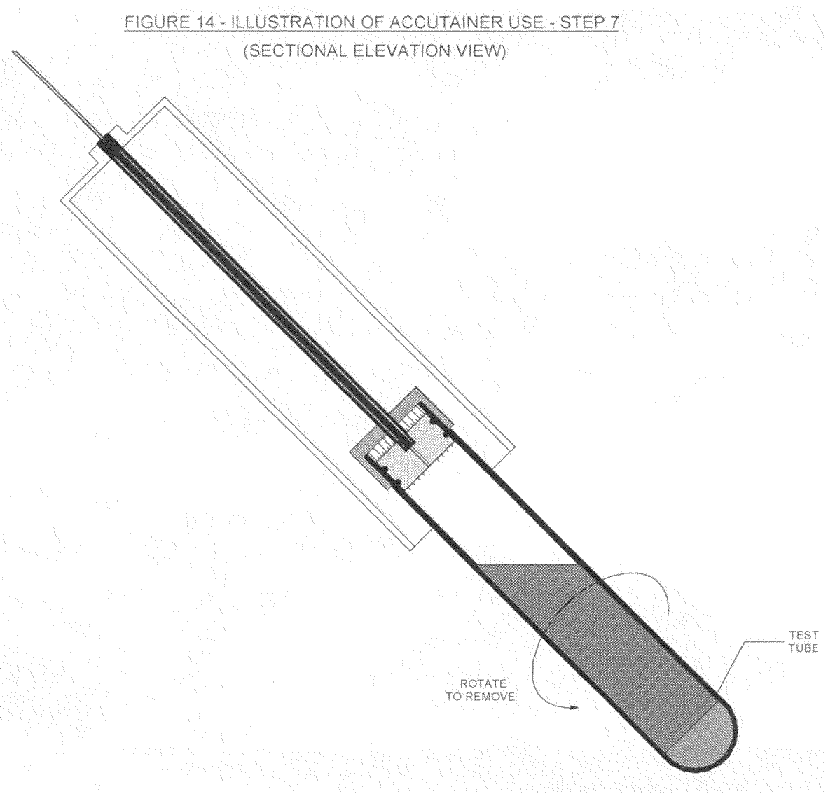

FIG. 14, step 7 illustrates that the piston in the test tube can be unthreaded from the plastic rod. The test tube can be rotated in the direction shown while holding the translucent protective holder in place. The ribs on the right side of the cap and the left side of the piston make the piston rotate when the test tube is rotated. Since the plastic rod is held in place by the translucent protective holder, the piston will be unthreaded and thereby separated from it.

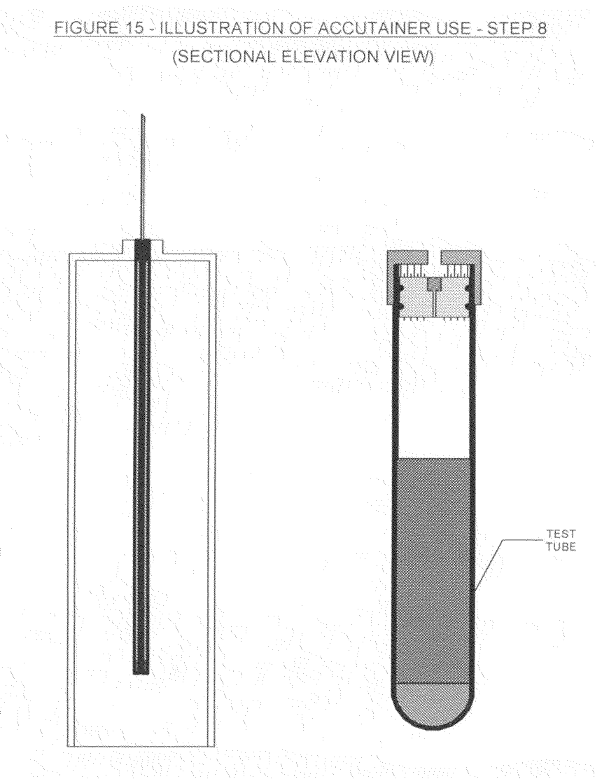

FIG. 15, step 8 illustrates that the test tube is now separate from the translucent protective holder and hypodermic needle. The test tube with the sample of blood can be sent to a laboratory for testing.

1. I claim a device that can be used to obtain a sample of blood from a blood vessel in a person or animal comprised of the following:
a hypodermic needle that is open to a blood vessel on one end, that penetrates a piston, as described below, and that is open to the opposite side of the piston at its other end
a piston that is in a fixed position relative to the hypodermic needle described above
a test tube or similar container with an inside diameter approximately equal to the outside diameter of the piston described above with the end nearest to the blood vessel open and the end farthest from the blood vessel closed, and that can be moved in a direction essentially parallel to the hypodermic needle
